(19) United States
(12) Patent Application Publication
(10) Pub. No.: US 2008/0269536 A1
(43) Pub. Date: Oct. 30, 2008

(54) Method for Producing Propene From Propane

(75) Inventors: Sven Crone, Limburgerhof (DE); Otto Machhammer, Mannheim (DE); Götz-Peter Schindler, Mannheim (DE); Frieder Borgmeier, Mannheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/817,599

(22) PCT Filed: Mar. 3, 2006
(86) PCT No.: PCT/EP06/60436

§ 371 Date: Aug. 31, 2007

(30) Foreign Application Priority Data
Mar. 8, 2005 (DE) 10 2005 010 586.6

(51) Int. Cl. C07C 5/48

(52) U.S. Cl. 585/655

(57) ABSTRACT

The invention relates to a process for preparing propene from propane, comprising the steps:

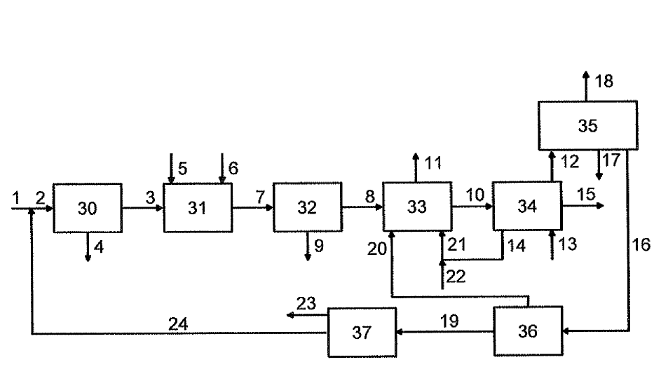

A) a feed gas stream a comprising propane is provided;

B) the feed gas stream a comprising propane, if appropriate steam and if appropriate and an oxygenous gas stream are fed into a dehydrogenation zone and propane is subjected to a dehydrogenation to propene to obtain a product gas stream b comprising propane, propene, methane, ethane, ethene, nitrogen, carbon monoxide, carbon dioxide, steam, if appropriate hydrogen and if appropriate oxygen;

C) the product gas stream b is cooled, if appropriate compressed and steam is removed by condensation to obtain a steam-depleted product gas stream c;

D) uncondensable or low-boiling gas constituents are removed by contacting the product gas stream c with an inert absorbent and subsequently desorbing the gases dissolved in the inert absorbent to obtain a C₃ hydrocarbon stream d1 and an offgas stream d2 comprising methane, ethane, ethene, nitrogen, carbon monoxide, carbon dioxide, if appropriate hydrogen and if appropriate oxygen;

E) the Ca hydrocarbon stream d1 is cooled and if appropriate compressed to obtain a gaseous or liquid Ca hydrocarbon stream e1;

F) the C₃ hydrocarbon stream e1 is if appropriate fed into a first distillation zone and separated distillatively into a stream f1 composed of propane and propene and a stream f2 comprising ethane and ethene;

G) the stream e1 or f1 is fed into a (second) distillation zone and separated distillatively into a product stream g1 composed of propene and a stream g2 composed of propane, the stream g2 being recirculated at least partially into the dehydrogenation zone.

The invention relates to a process for preparing propene from propane.

Propene is obtained on the industrial scale by dehydrogenating propane.

In the process, known as the UOP-oleflex process, for dehydrogenating propane to propene, a feed gas stream comprising propane is preheated to 600-700° C. and dehydrogenated in a moving bed dehydrogenation reactor over a catalyst which comprises platinum on alumina to obtain a product gas stream comprising predominantly propane, propene and hydrogen. In addition, low-boiling hydrocarbons formed by cracking (methane, ethane, ethene) and small amounts of high boilers (C₄⁺ hydrocarbons) are present in the product gas stream. The product gas mixture is cooled and compressed in a plurality of stages Subsequently, the C₂ and C₃ hydrocarbons and the high boilers are removed from the hydrogen and methane formed in the dehydrogenation by condensation in a “cold box”. The liquid hydrocarbon condensate is subsequently separated by distillation by removing the C₂ hydrocarbons and remaining methane in a first column and separating the C₃ hydrocarbon stream into a propene fraction having high purity and a propane fraction which also comprises the C₄⁺ hydrocarbons in a second distillation column.

A disadvantage of this process is the loss of C₃ hydrocarbons by the condensation in the cold box. Owing to the large amounts of hydrogen formed in the dehydrogenation and as a consequence of the phase equilibrium, relatively large amounts of C₃ hydrocarbons are also discharged with the hydrogen/methane offgas stream unless condensation is effected at very low temperatures. Thus, it is necessary to work at temperatures of from −20 to −60° C. in order to limit the loss of C₃ hydrocarbons which are discharged with the hydrogen/methane offgas stream.

It is an object of the present invention to provide an improved process for dehydrogenating propane to propene.

The object is achieved by a process for preparing propene from propane, comprising the steps:
A) a feed gas stream a comprising propane is provided;
B) the feed gas stream a comprising propane, if appropriate an oxygenous gas stream and if appropriate steam are fed into a dehydrogenation zone and propane is subjected to a dehydrogenation to propene to obtain a product gas stream b comprising propane, propene, methane, ethane, ethene, carbon monoxide, carbon dioxide, steam, if appropriate hydrogen and if appropriate to oxygen;
C) the product gas stream b is cooled, if appropriate compressed and steam is removed by condensation to obtain a steam-depleted product gas stream c;
D) uncondensable or low-boiling gas constituents are removed by contacting the product gas stream c with an inert absorbent and subsequently desorbing the gases dissolved in the inert absorbent to obtain a C₃ hydrocarbon stream d1 and an offgas stream d2 comprising methane, ethane, ethene, nitrogen, carbon monoxide, carbon dioxide, if appropriate hydrogen, if appropriate oxygen and if appropriate propane and propene;
E) the C₃ hydrocarbon stream d1 is cooled and if appropriate compressed to obtain a gaseous or liquid C₃ hydrocarbon stream e1;
F) the C₃ hydrocarbon stream e1 is if appropriate fed into a first distillation zone and separated distillatively into a stream f1 composed of propane and propene and a stream f2 comprising ethane and ethene;
G) the stream e1 or f1 is fed into a (second) distillation zone and separated distillatively into a product stream g1 composed of propene and a stream g2 composed of propane, the stream g2 being recycled at least partly into the dehydrogenation zone.

In a first process part, A), a feed gas stream a comprising propane is provided. This generally comprises at least 80% by volume of propane, preferably 90% by volume of propane. In addition, the propane-containing feed gas stream A generally also comprises butanes (n-butane, isobutane). Typical compositions of the propane-containing feed gas stream are disclosed in DE-A 102 46 119 and DE-A 102 45 585. Typically, the propane-containing feed gas stream a is obtained from liquid petroleum gas (LPG). To remove the butanes, the propane-containing feed gas stream can be subjected to a purification distillation to obtain a feed gas stream a with a very high propane content (>95% by volume).

In one process part, B), the feed gas stream comprising propane is fed into a dehydrogenation zone and subjected to a generally catalytic dehydrogenation. In this process part, propane is dehydrogenated partially in a dehydrogenation reactor over a dehydrogenation-active catalyst to give propene. In addition, hydrogen and small amounts of methane, ethane, ethene and C₄⁺ hydrocarbons (n-butane, isobutane, butenes, butadiene) are obtained. Also generally obtained in the product gas mixture of the catalytic propane dehydrogenation are carbon oxides (CO, CO₂), in particular CO₂, steam and if appropriate inert gases to a small degree. The product gas stream of the dehydrogenation generally comprises steam which has already been added to the dehydrogenation gas mixture and/or, in the case of dehydrogenation in the presence of oxygen (oxidative or non-oxidative), has already formed in the dehydrogenation. When the dehydrogenation is carried out in the presence of oxygen, inert gases (nitrogen) are introduced into the dehydrogenation zone with the oxygen-containing gas stream fed in, as long as pure oxygen is not fed in. In addition, unconverted propane is present in the product gas mixture.

The propane dehydrogenation can in principle be carried out in any reactor types known from the prior art. A comparatively comprehensive description of reactor types suitable in accordance with the invention is also contained in “Catalytica® Studies Division, Oxidative Dehydrogenation and Alternative Dehydrogenation Processes” (Study Number 4192 OD, 1993, 430 Ferguson Drive, Mountain View, Calif., 94043-5272, USA).

The dehydrogenation can be carried out as an oxidative or non-oxidative dehydrogenation. The dehydrogenation can be carried out isothermally or adiabatically. The dehydrogenation can be carried out catalytically in a fixed bed, moving bed or fluidized bed reactor.

The nonoxidative catalytic propane dehydrogenation can be carried out autothermally with oxygen being fed in. However, it can also be carried out purely catalytically without oxygen being fed in. In the autothermal dehydrogenation, oxygen is additionally admixed with the reaction gas mixture of the propane dehydrogenation in at least one reaction zone and the hydrogen and/or hydrocarbon present in the reaction gas mixture is at least partly combusted, which directly generates in the reaction gas mixture at least some of the heat required for dehydrogenation in the at least one reaction zone. The oxygen-comprising gas used is air or oxygen-enriched air with an oxygen content of up to 70% by volume, preferably up to 50% by volume.

One feature of the nonoxidative method compared to an oxidative method is that free hydrogen is still present at the outlet of the dehydrogenation zone. In the oxidative dehydrogenation, free hydrogen is present at the outlet of the dehydrogenation zone.

A suitable reactor form is the fixed bed tubular or tube bundle reactor. In these reactors, the catalyst (dehydrogenation catalyst and if appropriate a specialized oxidation catalyst) is disposed as a fixed bed in a reaction tube or in a bundle of reaction tubes. Customary reaction tube internal diameters in the non-autothermal, purely catalytic dehydrogenation are from about 10 to 15 cm. A typical dehydrogenation tube bundle reactor comprises from about 300 to 1000 reaction tubes. The internal temperature in the reaction tubes typically varies in the range from 300 to 1200° C., preferably in the range from 500 to 1000° C. The working pressure is customarily from 0.5 to 12 bar, frequently from 1 to 8 bar, when a low steam dilution is used, or else from 3 to 8 bar when a high steam dilution is used (corresponding to the steam active reforming process (STAR process) or the Linde process) for the dehydrogenation of propane or butane of Phillips Petroleum Co. Typical gas hourly space velocities (GHSV) are from 500 to 2000 h⁻¹, based on hydrocarbon used. The catalyst geometry may, for example, be spherical or cylindrical (hollow or solid).

The propane dehydrogenation may also be carried out under heterogeneous catalysis in a fluidized bed, according to the Snamprogetti/Yarsintez-FBD process. Appropriately, two fluidized beds are operated in parallel, of which one is generally in the state of regeneration. The working pressure is typically from 1 to 2 bar, the dehydrogenation temperature generally from 550 to 650° C. The heat required for the dehydrogenation can be introduced into the reaction system by preheating the dehydrogenation catalyst to the reaction temperature. The admixing of a cofeed comprising oxygen allows the preheater to be dispensed with and the required heat to be generated directly in the reactor system by combustion of hydrogen and/or hydrocarbons in the presence of oxygen. If appropriate, a cofeed comprising hydrogen may additionally be admixed.

The propane dehydrogenation can be carried out in a tray reactor. When the dehydrogenation is carried out autothermally with an oxygenous gas stream being fed in, it is preferably carried out in a tray reactor. This reactor comprises one or more successive catalyst beds. The number of catalyst beds may be from 1 to 20, advantageously from 1 to 6, preferably from 1 to 4 and in particular from 1 to 3. The catalyst beds are preferably flowed through radially or axially by the reaction gas. In general, such a tray reactor is operated using a fixed catalyst bed. In the simplest case, the fixed catalyst beds are disposed axially in a shaft furnace reactor or in the annular gaps of concentric cylindrical grids. A shaft furnace reactor corresponds to a tray reactor with only one tray. The performance of the dehydrogenation in a single shaft furnace reactor corresponds to one embodiment. In a further, preferred embodiment, the dehydrogenation is carried out in a tray reactor having 3 catalyst beds.

In general, the amount of the oxygenous gas added to the reaction gas mixture is selected in such a way that the amount of heat required for the dehydrogenation of the propane is generated by the combustion of the hydrogen present in the reaction gas mixture and of any hydrocarbons present in the reaction gas mixture and/or of carbon present in the form of coke. In general, the total amount of oxygen supplied, based on the total amount of propane, is from 0.001 to 0.5 mol/mol, preferably from 0.001 to 0.4 mol/mol, more preferably from 0.02 to 0.35 mol/mol. Oxygen may be used in the form of oxygenous gas which comprises inert gases, for example air or oxygen-enriched air.

The hydrogen combusted to generate heat is the hydrogen formed in the catalytic propane dehydrogenation and also any hydrogen additionally added to the reaction gas mixture as hydrogenous gas. The amount of hydrogen present should preferably be such that the molar H₂/O₂ ratio in the reaction gas mixture immediately after the oxygenous gas is fed in is from 1 to 10 mol/mol, preferably from 2 to 5 mol/mol. In multistage reactors, this applies to every intermediate feed of oxygenous and any hydrogenous gas.

The hydrogen is combusted catalytically. The dehydrogenation catalyst used generally also catalyzes both the combustion of the hydrocarbons and of hydrogen with oxygen, so that in principle no specialized oxidation catalyst is required apart from it. In one embodiment, operation is effected in the presence of one or more oxidation catalysts which selectively catalyze the combustion of hydrogen with oxygen to give water in the presence of hydrocarbons. The combustion of these hydrocarbons with oxygen to give CO, CO₂ and water therefore proceeds only to a minor extent. The dehydrogenation catalyst and the oxidation catalyst can be present together in one or more reaction zones or separately in different reaction zones.

When the reaction is carried out in more than one stage, the oxidation catalyst may be present only in one, in more than one or in all reaction zones.

Preference is given to disposing the catalyst which selectively catalyzes the oxidation of hydrogen at the points where there are higher partial oxygen pressures than at other points in the reactor, in particular near the feed point for the oxygenous gas. The oxygenous gas and/or hydrogenous gas may be fed in at one or more points in the reactor.

In one embodiment of the process according to the invention, there is intermediate feeding of oxygenous gas and if appropriate of hydrogenous gas upstream of each tray of a tray reactor. In a further embodiment of the process according to the invention, oxygenous gas and if appropriate hydrogenous gas are fed in upstream of each tray except the first tray. In one embodiment, a layer of a specialized oxidation catalyst is present downstream of every feed point, followed by a layer of the dehydrogenation catalyst. In a further embodiment, no specialized oxidation catalyst is present. The dehydrogenation temperature is generally from 400 to 1100° C.; the pressure in the last catalyst bed of the tray reactor is generally from 0.2 to 15 bar, preferably from 1 to 10 bar, more preferably from 1 to 5 bar. The GHSV is generally from 500 to 2000 h⁻¹, and, in high-load operation, even up to 100 000 h⁻¹, preferably from 4000 to 16 000 h⁻¹.

A preferred catalyst which selectively catalyzes the combustion of hydrogen comprises oxides and/or phosphates selected from the group consisting of the oxides and/or phosphates of germanium, tin, lead, arsenic, antimony and bismuth. A further preferred catalyst which catalyzes the combustion of hydrogen comprises a noble metal of transition group VIII and/or I of the periodic table.

The dehydrogenation catalysts used generally comprise a support and an active composition. The support generally consists of a heat-resistant oxide or mixed oxide. The dehydrogenation catalysts preferably comprise a metal oxide which is selected from the group consisting of zirconium dioxide, zinc oxide, aluminum oxide, silicon dioxide, titanium dioxide, magnesium oxide, lanthanum oxide, cerium oxide and mixtures thereof, as a support. The mixtures may be physical mixtures or else chemical mixed phases such as magnesium aluminum oxide or zinc aluminum oxide mixed oxides. Preferred supports are zirconium dioxide and/or silicon dioxide, and particular preference is given to mixtures of zirconium dioxide and silicon dioxide.

The active composition of the dehydrogenation catalysts generally comprises one or more elements of transition group VIII of the periodic table, preferably platinum and/or palladium, more preferably platinum. Furthermore, the dehydrogenation catalysts may comprise one or more elements of main group I and/or II of the periodic table, preferably potassium and/or cesium. The dehydrogenation catalysts may further comprise one or more elements of transition group III of the periodic table including the lanthanides and actinides, preferably lanthanum and/or cerium. Finally, the dehydrogenation catalysts may comprise one or more elements of main group III and/or IV of the periodic table, preferably one or more elements from the group consisting of boron, gallium, silicon, germanium, tin and lead, more preferably tin. Suitable shaped catalyst body geometries are extrudates, stars, rings, saddles, spheres, foams and monoliths with characteristic dimensions of from 1 to 100 mm.

In a preferred embodiment, the dehydrogenation catalyst comprises at least one element of transition group VIII, at least one element of main group I and/or II, at least one element of main group III and/or IV and at least one element of transition group III including the lanthanides and actinides.

For example, all dehydrogenation catalysts which are disclosed by WO 99/46039, U.S. Pat. No. 4,788,371, EP-A 705 136, WO 99/29420, U.S. Pat. No. 5,220,091, U.S. Pat. No. 5,430,220, U.S. Pat. No. 5,877,369, EP 0 117 146, DE-A 199 37 106, DE-A 199 37 105 and DE-A 199 37 107 may be used in accordance with the invention. Particularly preferred catalysts for the above-described variants of autothermal propane dehydrogenation are the catalysts according to examples 1, 2, 3 and 4 of DE-A 199 37 107.

Preference is given to carrying out the autothermal propane dehydrogenation in the presence of steam. The added steam serves as a heat carrier and supports the gasification of organic deposits on the catalysts, which counteracts carbonization of the catalysts and increases the onstream time of the catalysts. This converts the organic deposits to carbon monoxide and carbon dioxide. The dilution with steam shifts the equilibrium toward the products of the dehydrogenation.

The dehydrogenation catalyst may be regenerated in a manner known per se. For instance, steam may be added to the reaction gas mixture or a gas comprising oxygen may be passed from time to time over the catalyst bed at elevated temperature and the deposited carbon burnt off. After the regeneration, the catalyst is reduced with a hydrogenous gas if appropriate.

The product gas stream b may be separated into two substreams, in which case one substream is recycled into the autothermal dehydrogenation, corresponding to the cycle gas method described in DE-A 102 11 275 and DE-A 100 28 582.

The propane dehydrogenation may be carried out as an oxidative dehydrogenation. The oxidative propane dehydrogenation may be carried out as a homogeneous oxidative dehydrogenation or as a heterogeneously catalyzed oxidative dehydrogenation.

When the propane dehydrogenation in the process according to the invention is configured as a homogeneous oxydehydrogenation, this can in principle be carried out as described in the documents U.S. Pat. No. 3,798,283, CN-A 1,105,352, Applied Catalysis, 70 (2), 1991, p. 175 to 187, Catalysis Today 13, 1992, p. 673 to 678 and the prior application DE-A 1 96 22 331.

The temperature of the homogeneous oxydehydrogenation is generally from 300 to 700° C., preferably from 400 to 600° C., more preferably from 400 to 500° C. The pressure may be from 0.5 to 100 bar or from 1 to 50 bar. It will frequently be from 1 to 20 bar, in particular from 1 to 10 bar.

The residence time of the reaction gas mixture under oxydehydrogenation conditions is typically from 0.1 or 0.5 to 20 sec, preferably from 0.1 or 0.5 to 5 sec. The reactor used may, for example, be a tube bundle reactor such as a tube bundle reactor with salt melt as a heat carrier, or a shaft furnace reactor with intermediate cooling.

The propane to oxygen ratio in the starting mixture to be used may be from 0.5:1 to 40:1. The molar ratio of propane to molecular oxygen in the starting mixture is preferably≦6:1, more preferably≦5:1. In general, the aforementioned ratio will be≧1:1, for example≧2:1. The starting mixture may comprise further, substantially inert constituents such as H₂O, CO₂, CO, N₂, noble gases and/or propene. It is favorable for a homogeneous oxidative dehydrogenation of propane to propene when the ratio of the surface area of the reaction space to the volume of the reaction space is at a minimum. Particularly favorable surface materials are aluminas, quartz glass, borosilicates, stainless steel and aluminum.

When the first reaction stage in the process according to the invention is configured as a heterogeneously catalyzed oxydehydrogenation, this can in principle be carried out as described in the documents U.S. Pat. No. 4,788,371, CN-A 1073893 Catalysis Letters 23 (1994) 103-106, W. Zhang, Gaodeng Xuexiao Huaxue Xuebao, 14 (1993) 566, Z. Huang, Shiyou Huagong, 21 (1992) 592, WO 97/36849, DE-A 1 97 53 817, U.S. Pat. No. 3,862,256, U.S. Pat. No. 3,887,631, DE-A 1 95 30 454, U.S. Pat. No. 4,341,664, J. of Catalysis 167, 560-569 (1997), J. of Catalysis 167, 550-559 (1997), Topics in Catalysis 3 (1996) 265-275, U.S. Pat. No. 5,086,032, Catalysis Letters 10 (1991) 181-192, Ind. Eng. Chem. Res. 1996, 35, 14-18, U.S. Pat. No. 4,255,284, Applied Catalysis A: General, 100 (1993) 111-130, J. of Catalysis 148, 56-67 (1994), V. Cortés Corberán and S. Vic Bellón (Editors), New Developments in Selective Oxidation II, 1994, Elsevier Science B.V., p. 305-313, 3rd World Congress on Oxidation Catalysis R. K. Grasselli, S. T. Oyama, A. M. Gaffney and J. E. Lyons (Editors), 1997, Elsevier Science B.V., p. 375 ff. In particular, all of the oxydehydrogenation catalysts specified in the aforementioned documents may be used. The statement made for the abovementioned documents also applies to:
a) Otsuka, K.; Uragami, Y.; Komatsu, T.; Hatano, M. in Natural Gas Conversion, Stud. Surf. Sci. Catal.; Holmen A.; Jens, K.-J.; Kolboe, S., Eds.; Elsevier Science: Amsterdam, 1991; Vol. 61, p 15;
b) Seshan, K.; Swaan, H. M.; Smits, R. H. H.; van Ommen, J. G.; Ross, J. R. H. in New Developments in Selective Oxidation; Stud. Surf. Sci. Catal.; Centi, G.; Trifirò, F., Eds; Elsevier Science: Amsterdam 1990; Vol. 55, p 505;
c) Smits, R. H. H.; Seshan, K.; Ross, J. R. H. in New Developments in Selective Oxidation by Heterogeneous Catalysis; Stud. Surf. Sci. Catal; Ruiz, P.; Delmon, B., Eds.; Elsevier Science: Amsterdam, 1992 a; Vol. 72, p 221;
d) Smits, R. H. H.; Seshan, K.; Ross, J. R. H. Proceedings, Symposium on Catalytic Selective Oxidation, Washington DC; American Chemical Society: Washington, DC, 1992 b; 1121;
e) Mazzocchia, C.; Aboumrad, C.; Daigne, C.; Tempesti, E.; Herrmann, J. M.; Thomas, G. Catal. Lett. 1991, 10, 181;
f) Bellusi, G.; Conti, G.; Perathonar, S.; Trifirò, F. Proceedings, Symposium on Catalytic Selective Oxidation, Washington, DC; American Chemical Society: Washington, DC, 1992; p 1242;
g) Ind. Eng. Chem. Res. 1996, 35, 2137-2143 and
h) Symposium on Heterogeneous Hydrocarbon Oxidation Presented before the Division of Petroleum Chemistry, Inc. 211th National Meeting, American Chemical Society New Orleans, La., Mar. 24-29, 1996.

Particularly suitable oxydehydrogenation catalysts are the multimetal oxide compositions or catalysts A of DE-A 1 97 53 817, and the multimetal oxide compositions or catalysts A specified as preferred are very particularly favorable. In other words, useful active compositions are in particular multimetal oxide compositions of the general formula I

M¹ₐMO_{1-b}M²_{b}Oₓ (I)

where
m¹=Co, Ni, Mg, Zn, Mn and/or Cu,
M²=W, V, Te, Nb, P, Cr, Fe, Sb, Ce, Sn and/or La,
a=from 0.5 to 1.5,
b=from 0 to 0.5 and
x=a number which is determined by the valency and frequency of the elements in I other than oxygen.

Further multimetal oxide compositions suitable as oxydehydrogenation catalysts are specified below:

Suitable Mo—V—Te/Sb—Nb—O multimetal oxide catalysts are disclosed in EP-A 0 318 295, EP-A 0 529 853, EP-A 0 603 838, EP-A 0 608 836, EP-A 0 608 838, EP-A 0 895 809, EP-A 0 962 253, EP-A 1 192 987, DE-A 198 35 247, DE-A 100 51 419 and DE-A 101 19 933.

Suitable Mo—V—Nb—O multimetal oxide catalysts are described, inter alia, in E. M. Thorsteinson, T. P. Wilson, F. G. Young, P. H. Kasei, Journal of Catalysis 52 (1978), pages 116-132, and in U.S. Pat. No. 4,250,346 and EP-A 0 294 845.

Suitable Ni—X—O multimetal oxide catalysts where X=Ti, Ta, Nb, Co, Hf, W, Y, Zn, Zr, Al, are described in WO 00/48971.

In principle, suitable active compositions can be prepared in a simple manner by obtaining from suitable sources of their components a very intimate, preferably finely divided dry mixture corresponding to the stoichiometry and calcining it at temperatures of from 450 to 1000° C. The calcination may be effected either under inert gas or under an oxidative atmosphere, for example air (mixture of inert gas and oxygen), and also under a reducing atmosphere (for example mixture of inert gas, oxygen and NH₃, CO and/or H₂). Useful sources for the components of the multimetal oxide active compositions 1 include oxides and/or those compounds which can be converted to oxides by heating, at least in the presence of oxygen. In addition to the oxides, such useful starting compounds are in particular halides, nitrates, formates, oxalates, citrates, acetates, carbonates, amine complex salts, ammonium salts and/or hydroxides.

The multimetal oxide compositions may be used for the process according to the invention either in powder form or shaped to certain catalyst geometries, and this shaping may be effected before or after the final calcining. Suitable unsupported catalyst geometries are, for example, solid cylinders or hollow cylinders having an external diameter and a length of from 2 to 10 mm. In the case of the hollow cylinders, a wall thickness of from 1 to 3 mm is appropriate. The suitable hollow cylinder geometries are, for example, 7 mm×7 mm×4 mm or 5 mm×3 mm×2 mm or 5 mm×2 mm×2 mm (in each case length×external diameter×internal diameter). The unsupported catalyst can of course also have spherical geometry, in which case the sphere diameter may be from 2 to 10 mm.

The pulverulent active composition or its pulverulent precursor composition which is yet to be calcined may of course also be shaped by applying to preshaped inert catalyst supports. The layer thickness of the powder composition applied to the support bodies is appropriately selected within the range from 50 to 500 mm, preferably within the range from 150 to 250 mm. Useful support materials include customary porous or nonporous aluminum oxides, silicon dioxide, thorium dioxide, zirconium dioxide, silicon carbide or silicates such as magnesium silicate or aluminum silicate. The support bodies may have a regular or irregular shape, preference being given to regularly shaped support bodies having distinct surface roughness, for example spheres, hollow cylinders or saddles generally having dimensions in the range from 1 to 100 mm. It is suitable to use substantially nonporous, surface-rough, spherical supports of steatite whose diameter is from 1 to 8 mm, preferably from 4 to 5 mm.

The reaction temperature of the heterogeneously catalyzed oxydehydrogenation of propane is generally from 300 to 600° C., typically from 350 to 500° C. The pressure is from 0.5 to 10 bar, preferably from 1 to 10 bar, for example from 1 to 5 bar. Pressures above 1 bar, for example from 1.5 to 10 bar, have been found to be particularly advantageous. In general, the heterogeneously catalyzed oxydehydrogenation of propane is effected over a fixed catalyst bed. The latter is appropriately deposited in the tubes of a tube bundle reactor, as described, for example, in EP-A 700 893 and in EP-A 700 714 and the literature cited in these documents. The average residence time of the reaction gas mixture in the catalyst bed is normally from 0.5 to 20 sec. The propane to oxygen ratio in the starting reaction gas mixture to be used for the heterogeneously catalyzed propane oxydehydrogenation may, according to the invention, be from 0.5:1 to 40:1. It is advantageous when the molar ratio of propane to molecular oxygen in the starting gas mixture is≦6:1, preferably≦5:1. In general, the aforementioned ratio is≧1:1, for example 2:1. The starting gas mixture may comprise further, substantially inert constituents such as H₂O, CO₂, CO, N₂, noble gases and/or propene. In addition, C₁, C₂ and C₄ hydrocarbons may also be comprised to a small extent.

In the propane dehydrogenation, a gas mixture is obtained which generally has the following composition: from 5 to 95% by volume of propane, from 1 to 50% by volume of propene, from 0 to 20% by volume of methane, ethane, ethene and C₄⁺ hydrocarbons, from 0 to 30% by volume of carbon oxides, from 0 to 70% by volume of steam, from 0 to 30% by volume of hydrogen, and from 0 to 70% by volume of inert gases.

In the autothermal propane dehydrogenation, a gas mixture is obtained which generally has the following composition: from 10 to 80% by volume of propane, from 1 to 40% by volume of propene, from 0 to 20% by volume of methane, ethane, ethene and C₄⁺ hydrocarbons, from 0.1 to 30% by volume of carbon oxides, from 0.1 to 70% by volume of steam, from 1 to 30% by volume of hydrogen, and also from 0 to 50% by volume of inert gases (in particular nitrogen).

When it leaves the dehydrogenation zone, the product gas stream b is generally under a pressure of from 1 to 20 bar, preferably from 1 to 10 bar, more preferably from 1 to 5 bar, and has a temperature in the range from 400 to 700° C.

In process part C), steam is initially removed from the product gas stream b to obtain a steam-depleted product gas stream c. The removal of steam is carried out by condensation, by cooling and if appropriate preceding compression of the product gas stream b, and may be carried out in one or more cooling and if appropriate compression stages. In general, the product gas stream b is cooled for this purpose to a temperature in the range from 0 to 80° C., preferably from 10 to 65° C. In addition, the product gas stream may be compressed, for example to a pressure in the range from 2 to 40 bar, preferably from 5 to 20 bar, more preferably from 10 to 20 bar.

In one embodiment of the process according to the invention, the product gas stream b is passed through a battery of heat exchangers and initially thus initially cooled to a temperature in the range from 50 to 200° C. and subsequently cooled further in a quenching tower with water to a temperature of from 40 to 80° C. for example 55° C. This condenses out the majority of the steam, but also some of the C₄⁺ hydrocarbons present in the product gas stream b, in particular the C₅⁺ hydrocarbons. Suitable heat exchangers are, for example, direct heat exchangers and countercurrent heat exchangers, such as gas-gas countercurrent heat exchangers, and air coolers.

A steam-depleted product gas stream c is obtained. This generally still comprises from 0 to 10% by volume of steam. For the virtually full removal of water from the product gas stream c when certain solvents are used in step D), a drying by means of molecular sieve or membranes may be provided.

In one process part, D), the uncondensable or low-boiling gas constituents such as hydrogen, oxygen, carbon monoxide, carbon dioxide, nitrogen and the low-boiling hydrocarbons (methane, ethane, ethene) are removed from the C₃-hydrocarbons by means of a high-boiling absorbent in an absorption/desorption cycle to obtain a stream dl which comprises the C₃ hydrocarbons and additionally also small amounts of ethane and ethene, and an offgas stream d2 which comprises the uncondensable or low-boiling gas constituents.

To this end, in an absorption stage, the gas stream c is contacted with an inert absorbent and the C₃ hydrocarbons and also small amounts of the C₂ hydrocarbons are absorbed in the inert absorbent to obtain an absorbent laden with C₃ hydrocarbons and an offgas d2 comprising the remaining gas constituents. These are essentially carbon oxides, hydrogen, inert gases, and also C₂ hydrocarbons and methane. Certain amounts of propane and propene may also be comprised in the stream d2, since the removal is generally not entirely complete. In a desorption stage, the C₃ hydrocarbons are released again from the absorbent.

Inert absorbents used in the absorption stage are generally high-boiling nonpolar solvents in which the C₃ hydrocarbon mixture to be removed has a distinctly higher solubility than the remaining gas substituents to be removed. The absorption can be effected by simply passing stream c through the absorbent. However, it can also be effected in columns. It is possible to work in cocurrent, countercurrent or crosscurrent. Suitable absorption columns are, for example, tray columns having bubble-cap, valve and/or sieve trays, columns having structured packings, for example fabric packings or sheet metal packings having a specific surface area of from 100 to 1000 m²/m³, such as Mellapak® 250 Y, and columns having random packing, for example having spheres, rings or saddles made of metal, plastic or ceramic as random packings. However, useful absorption apparatus also includes trickle and spray towers, graphite block absorbers, surface absorbers such as thick-film and thin-film absorbers, and bubble columns with and without internals.

The absorption column preferably has an absorption section and a rectification section. To increase the enrichment of C₃ hydrocarbons in the solvent by the method of a rectification, it is then possible to introduce heat into the column bottom. Alternatively, a stripping gas stream can be fed into the column bottom, for example composed of nitrogen, air, steam or propane/propene mixtures. Thus, the laden absorbent is contacted with the stripping gas stream in the rectification section of the absorption column. This strips C₂⁻ hydrocarbons out of the laden absorbent. A portion of the top product can be condensed and introduced as reflux back to the top of the column in order to restrict solvent losses.

Suitable absorbents are comparatively nonpolar organic solvents, for example aliphatic C₄-C₁₈-alkenes, naphtha or aromatic hydrocarbons such as the middle oil fractions from paraffin distillation, or ethers having bulky groups, or mixtures of these solvents, to each of which a polar solvent such as dimethyl 1,2-phthalate may be added. Further suitable absorbents are esters of benzoic acid and phthalic acid with straight-chain C₁-C₈-alkanols, such as n-butyl benzoate, methyl benzoate, ethyl benzoate, dimethyl phthalate, diethyl phthalate, and also heat carrier oils such as biphenyl and diphenyl ether, their chlorine derivatives, and also triarylalkenes. A suitable absorbent is a mixture of biphenyl and diphenyl ether, preferably in the azeotropic composition, for example the commercially available Diphyl®. This solvent mixture frequently comprises dimethyl phthalate in an amount of from 0.1 to 25% by weight. Suitable absorbents are also butanes, pentanes, hexanes, heptanes, octanes, nonanes, decanes, undecanes, dodecanes, tridecanes, tetradecanes, pentadecanes, hexadecanes, heptadecanes and octadecanes, or fractions which are obtained from refinery streams and comprise the linear alkanes mentioned as main components. Preferred absorbents are C₈-C₁₀ hydrocarbons; particular preference is given to C₉ hydrocarbons, in particular nonanes.

To desorb the C₃ hydrocarbons, the laden absorbent is heated and/or decompressed to a lower pressure. Alternatively, the desorption can also be effected by stripping, typically with steam, or in a combination of decompression, heating and stripping, in one or more process steps. For example, the desorption may be carried out in two stages, in which case the second desorption stage is carried out at a lower pressure than the first desorption stage and the desorption gas of the second stage is recycled into the absorption stage. The absorbent regenerated in the desorption stage is recycled into the absorption stage. If appropriate, a portion of this absorbent stream which may comprise C₄⁺ hydrocarbons is discharged, worked up and recycled, or discarded.

In one process variant, the desorption step is carried out by decompressing and/or heating the laden absorbent. In a further process variant, stripping is effected additionally with steam.

In a further process variant, the absorbent laden with C₃ hydrocarbons, before the desorption, is compressed to a pressure which is needed in one of the following stages, for example the propane/propene separation (step G).

The removal D) is generally not entirely complete, so that, depending on the type of removal, small amounts or even only traces of the further gas constituents, in particular of the low-boiling hydrocarbons, may be present in the C₃ hydrocarbon stream d1.

To remove the hydrogen comprised in the offgas stream d2, it may, if appropriate after it has been cooled, for example, be passed in an indirect heat exchanger through a membrane, generally configured as a tube, which is permeable only to molecular hydrogen. The thus removed molecular hydrogen may, if required, be used at least partly in the dehydrogenation or else sent to another utilization, for example to the generation of electrical energy in fuel cells. Alternatively, the offgas stream d2 may be incinerated.

In one process part, E), the gas stream d1 is cooled, and it may additionally be compressed in one or more further compression stages. This affords a gaseous C₃ hydrocarbon stream e1 or a liquid condensate stream e1 composed of CS hydrocarbons. The stream e1 may still comprise small amounts of C₂ hydrocarbons. In addition, an aqueous condensate stream e2 and, if appropriate, small amounts of an offgas stream e3 may be obtained. The aqueous condensate stream e2 is generally obtained when the dissolved gases are stripped in step D) with steam for desorption.

The compression can in turn be effected in one or more stages. In general, compression is effected overall from a pressure in the range from 1 to 29 bar, preferably from 1 to 10 bar, to a pressure in the range from 12 to 30 bar. Each compression stage is followed by a cooling stage in which the gas stream is cooled to a temperature in the range from 15 to 80° C., preferably from 15 to 60° C. Subsequently, the compressed gas mixture is cooled to a temperature of from −10° C. to 60° C., preferably from −10° C. to 30° C. The liquid condensate streams e1 and e2 are separated from one another in a phase separation apparatus.

However, the gas stream d1 may also only be cooled, preferably when the dissolved gases are desorbed in process part D) at high pressure.

In one process part, F), the gaseous or liquid C₃ hydrocarbon stream e1 is fed into a first distillation zone and separated distillatively into a stream f1 comprising the C₃ hydrocarbons propane and propene, and a stream f2 comprising the C₂ hydrocarbons ethane and ethene. To this end, the C₃ hydrocarbon stream e1 is generally fed into a C2/C3 separating column having typically from 20 to 80 theoretical plates, for example approx. 60 theoretical plates. This column is generally operated at a pressure in the range from 10 to 30 bar, for example at approx. 20 bar, and a reflux ratio of 2-70. The bottom temperature is generally from 40 to 100° C., for example approx. 60° C., the top temperature from −20 to 10° C., for example approx. 10° C.

A stream f1 composed of propane and propene is obtained as the bottom draw stream having a total ethane/ethene content of generally<5000 ppm, preferably<1000 ppm, more preferably<500 ppm. The stream f2, which is preferably obtained as the top draw stream, may still comprise certain amounts of propane and propene and may be recycled to the absorption stage for removal thereof.

The process part F) may also be dispensed with, in particular when the stream d1 or e1 only has a low content of C₂ hydrocarbons.

In one process part, G), the C₃ hydrocarbon stream e1 or f1 is fed into a second distillation zone and separated distillatively into a stream g1 comprising propene and a stream g2 comprising propane. To this end, the hydrocarbon stream f1 is generally fed into a C₃ separating column (C3 splitter) having typically from 80 to 150 theoretical plates, for example approx. 100 theoretical plates. This column is generally operated at a pressure in the range from 10 to 30 bar, for example at approx. 250 bar, and a reflux ratio of 2-50. The bottom temperature is generally from 40 to 100° C., for example approx. 68° C., the top temperature from 30 to 60° C., for example approx. 60° C. Instead of a single C₃ separating column, two C₃ separating columns can also be used, in which case the first column is operated at relatively high pressure, for example 25 bar, and the second column is operated at relatively low pressure, for example 18 bar (2-column method). The top draw from the first column is liquefied in the bottom heater of the second column, and the bottom draw from the first column is fed into the second column. Alternatively, a method with vapor compression is also possible.

In one process part, H), the stream g2 and a fresh propane stream can be fed into a third distillation zone, in which a stream comprising C₄⁺ hydrocarbons is removed distillatively and the feed gas stream a is obtained with a very high propane content. The recycled stream g2 is in this case evaporated before entry into the third distillation zone. This can provide a coolant stream which can be used for cooling at another point, for example for cooling at the top of the C2/C3 separating column.

The invention is illustrated in detail by the example which follows.

### EXAMPLE

The variant, shown in the figure, of the process according to the invention was simulated by calculation. The process parameters below were assumed.

A capacity of the plant of 348 kt/a of propene at running time 8000 h, corresponding to 43 447 kg/h of propene, is assumed.

In addition to 98% by weight of propane, the fresh propane stream 1 comprises approx. 2% by weight of butane. The fresh propane stream 1 is mixed with the propane recycle stream 24 from the C3 splitter 37 and fed to the C3/C4 separating column 30. In the C3/C4 separating column 30 which has 40 theoretical plates and is operated at an operating pressure of 10 bar and a reflux ratio of 0.4, a high boiling stream 4 is removed and a propane stream 3 having a butane content of only 0.01% by weight is thus obtained. The propane stream 3 is preheated to 450° C., enters the dehydrogenation zone 31 and is subjected to an autothermal dehydrogenation. To this end, an oxygenous gas 6 and steam 5 are fed into the dehydrogenation zone 31. The conversion of the dehydrogenation is, based on propane, 40%; the selectivity of propene formation is 90%. In addition, 5% cracking products and 5% carbon oxides are formed by total combustion. The water concentration in the exit gas of the dehydrogenation zone is approx. 11% by weight; the residual oxygen content in the exit gas is 0% by weight; the exit temperature of the product gas mixture is 600° C. The product gas stream 7 is cooled and compressed in the compressor 32 starting from a pressure of 2.0 bar in 3 stages to a pressure of 15 bar. After the first and the second compressor stage, cooling is effected in each case to 55° C. This affords an aqueous condensate 9 which is discharged from the process. The compressed and cooled gas stream 8 is contacted in the absorption column 33 with tetradecane 21 as the absorbent. The unabsorbed gases are drawn off as offgas stream 11 via the top of the column; the absorbent laden with the C₃ hydrocarbons is withdrawn via the bottom of the column and fed to the desorption column 34. In the desorption column 34, decompression to a pressure of 4 bar and stripping with high-pressure steam fed as stream 13 desorbs the C₃ hydrocarbons to obtain a stream 14 composed of regenerated absorbent and a stream 12 composed of C₃ hydrocarbons and steam. The regenerated absorbent 14 is supplemented by fresh absorbent 22 and recycled into the absorption column 33. At the top of the desorption column, the gas is cooled to 45° C., which condenses out further absorbent 14. Also obtained is an aqueous phase which is removed in a phase separator and discharged from the process as stream 15. Subsequently, the stream 12 is compressed in two stages to a pressure of 16 bar and cooled to a temperature of 40° C. This affords a small offgas stream 18, a wastewater stream 17 and a liquid C₃ hydrocarbon stream 16.

A C₂ hydrocarbon stream 20 which additionally comprises certain amounts of C₃ hydrocarbons is removed from the liquid C₃ hydrocarbon stream 16 via the top of a C2/C3 separating column 36 having 30 theoretical plates at 16 bar and a reflux ratio of 63. The stream 20 is recycled into the absorption column 33, where C₃ hydrocarbons comprised in the stream 20 are removed The bottom temperature in the C2/C3 separating column 36 is 42° C., the top temperature −4° C. The residual ethane content of the bottom draw stream 19 is 0.01% by weight The bottom draw stream 19 is fed to a propane/propene separating column having 120 theoretical plates, which is operated at 16 bar with a reflux ratio of 22. The bottom temperature is 46° C., the top temperature 38° C. At the top, a propene stream 23 having a purity of 99.5% by weight of propene is obtained. The bottom draw stream 24 comprises approx. 98.5% by weight of propane and is recycled into the dehydrogenation zone 31.

TABLE 1

| Stream No. | 1 | 2 | 3 | 4 | 5 | 6 | 7 | 8 |
|---|---|---|---|---|---|---|---|---|
| Amount [kg/h] | 55060 | 133873 | 132763 | 1110 | 13157 | 79714 | 225633 | 200180 |
| BUTANE | 0.0200 | 0.0083 | 0.0001 | 0.9885 | 0.0000 | 0.0000 | 0.0001 | 0.0001 |
| PROPANE | 0.9800 | 0.9829 | 0.9910 | 0.0100 | 0.0000 | 0.0000 | 0.3499 | 0.3943 |
| PROPENE | 0.0000 | 0.0088 | 0.0089 | 0.0000 | 0.0000 | 0.0000 | 0.2056 | 0.2317 |
| WATER | 0.0000 | 0.0000 | 0.0000 | 0.0000 | 1.0000 | 0.0000 | 0.1140 | 0.0016 |
| ETHENE | 0.0000 | 0.0000 | 0.0000 | 0.0000 | 0.0000 | 0.0000 | 0.0037 | 0.0042 |
| ETHANE | 0.0000 | 0.0000 | 0.0000 | 0.0000 | 0.0000 | 0.0000 | 0.0080 | 0.0089 |
| TDK | 0.0000 | 0.0000 | 0.0000 | 0.0015 | 0.0000 | 0.0000 | 0.0000 | 0.0000 |
| CO2 | 0.0000 | 0.0000 | 0.0000 | 0.0000 | 0.0000 | 0.0000 | 0.0233 | 0.0262 |
| H2 | 0.0000 | 0.0000 | 0.0000 | 0.0000 | 0.0000 | 0.0000 | 0.0055 | 0.0062 |
| O2 | 0.0000 | 0.0000 | 0.0000 | 0.0000 | 0.0000 | 0.2000 | 0.0000 | 0.0000 |
| N2 | 0.0000 | 0.0000 | 0.0000 | 0.0000 | 0.0000 | 0.8000 | 0.2826 | 0.3186 |
| CO | 0.0000 | 0.0000 | 0.0000 | 0.0000 | 0.0000 | 0.0000 | 0.0074 | 0.0084 |
| Temperature [° C.] | 20.0 | 30.0 | 26.9 | 78.2 | 350.0 | 600.0 | 600.0 | 30.0 |
| Pressure [bar] | 10.0 | 10.0 | 10.0 | 10.0 | 3.0 | 3.0 | 2.4 | 14.8 |

| Stream No. | 9 | 10 | 11 | 12 | 13 | 14 | 15 | 16 |
|---|---|---|---|---|---|---|---|---|
| Amount [kg/h] | 25452 | 923770 | 77827 | 124294 | 10000 | 800668 | 8808 | 122983 |
| BUTANE | 0.0001 | 0.0000 | 0.0000 | 0.0001 | 0.0000 | 0.0000 | 0.0002 | 0.0001 |
| PROPANE | 0.0001 | 0.0843 | 0.0145 | 0.6262 | 0.0000 | 0.0000 | 0.0002 | 0.6326 |
| PROPENE | 0.0002 | 0.0487 | 0.0249 | 0.3584 | 0.0000 | 0.0005 | 0.0002 | 0.3620 |
| WATER | 0.9988 | 0.0004 | 0.0030 | 0.0104 | 1.0000 | 0.0003 | 0.9979 | 0.0004 |
| ETHENE | 0.0000 | 0.0001 | 0.0108 | 0.0011 | 0.0000 | 0.0000 | 0.0000 | 0.0011 |
| ETHANE | 0.0009 | 0.0005 | 0.0225 | 0.0038 | 0.0000 | 0.0000 | 0.0006 | 0.0038 |
| TDK | 0.0000 | 0.8660 | 0.0000 | 0.0001 | 0.0000 | 0.9991 | 0.0009 | 0.0000 |
| CO2 | 0.0000 | 0.0000 | 0.0675 | 0.0000 | 0.0000 | 0.0000 | 0.0000 | 0.0000 |
| H2 | 0.0000 | 0.0000 | 0.0159 | 0.0000 | 0.0000 | 0.0000 | 0.0000 | 0.0000 |
| O2 | 0.0000 | 0.0000 | 0.0000 | 0.0000 | 0.0000 | 0.0000 | 0.0000 | 0.0000 |
| N2 | 0.0000 | 0.0000 | 0.8194 | 0.0000 | 0.0000 | 0.0000 | 0.0000 | 0.0000 |
| CO | 0.0000 | 0.0000 | 0.0215 | 0.0000 | 0.0000 | 0.0000 | 0.0000 | 0.0000 |
| Temperature [° C.] | 54.2 | 49.7 | 36.0 | 45.0 | 264.0 | 150.0 | 45.0 | 41.5 |
| Pressure [bar] | 14.8 | 14.8 | 14.8 | 3.9 | 50.0 | 4.0 | 3.9 | 16.1 |

| Stream No. | 17 | 18 | 19 | 20 | 21 | 22 | 23 | 24 |
|---|---|---|---|---|---|---|---|---|
| Amount [kg/h] | 1261 | 50 | 122260 | 723 | 800694 | 26 | 43447 | 78813 |
| BUTANE | 0.0004 | 0.0000 | 0.0001 | 0.0000 | 0.0000 | 0.0000 | 0.0000 | 0.0001 |
| PROPANE | 0.0025 | 0.5723 | 0.6361 | 0.0354 | 0.0000 | 0.0000 | 0.0035 | 0.9849 |
| PROPENE | 0.0016 | 0.4061 | 0.3633 | 0.1500 | 0.0005 | 0.0000 | 0.9950 | 0.0150 |
| WATER | 0.9819 | 0.0021 | 0.0004 | 0.0000 | 0.0003 | 0.0000 | 0.0012 | 0.0000 |
| ETHENE | 0.0000 | 0.0065 | 0.0000 | 0.1894 | 0.0000 | 0.0000 | 0.0000 | 0.0000 |
| ETHANE | 0.0032 | 0.0130 | 0.0001 | 0.6252 | 0.0000 | 0.0000 | 0.0003 | 0.0000 |
| TDK | 0.0105 | 0.0000 | 0.0000 | 0.0000 | 0.9991 | 1.0000 | 0.0000 | 0.0000 |
| CO2 | 0.0000 | 0.0000 | 0.0000 | 0.0000 | 0.0000 | 0.0000 | 0.0000 | 0.0000 |
| H2 | 0.0000 | 0.0000 | 0.0000 | 0.0000 | 0.0000 | 0.0000 | 0.0000 | 0.0000 |
| O2 | 0.0000 | 0.0000 | 0.0000 | 0.0000 | 0.0000 | 0.0000 | 0.0000 | 0.0000 |
| N2 | 0.0000 | 0.0000 | 0.0000 | 0.0000 | 0.0000 | 0.0000 | 0.0000 | 0.0000 |
| CO | 0.0000 | 0.0000 | 0.0000 | 0.0000 | 0.0000 | 0.0000 | 0.0000 | 0.0000 |
| Temperature [° C.] | 35.0 | 41.5 | 42.2 | −3.6 | 35.0 | 35.0 | 38.2 | 46.4 |
| Pressure [bar] | 16.1 | 16.1 | 16.0 | 16.0 | 14.8 | 14.8 | 15.9 | 15.9 |

1-15. (canceled)

16. A process for preparing propene from propane, comprising:
A) providing a feed gas stream (a) comprising propane;
B) feeding said feed gas stream (a), and optionally steam and an oxygenous gas stream, into a dehydrogenation zone and dehydrogenating said propane of said feed gas stream (a) to propene to obtain a product gas stream (b) comprising propane, propene, methane, ethane, ethene, nitrogen, carbon monoxide, carbon dioxide, steam, and optionally hydrogen and/or oxygen;
C) cooling, and optionally compressing, said product gas stream (b) and removing steam from said product gas stream (b) via condensation to obtain a steam-depleted product gas stream (c);
D) contacting said product gas stream (c) with an inert absorbent and subsequently desorbing the gases dissolved in said inert absorbent by stripping with steam to remove uncondensable or low-boiling gas constituents from said product gas stream (c) and to obtain a C₃ hydrocarbon stream (d1) and an offgas stream (d2) comprising methane, ethane, ethene, nitrogen, carbon monoxide, carbon dioxide, and optionally hydrogen and/or oxygen;
E) cooling, and optionally compressing, said C₃ hydrocarbon stream (d1) to obtain a gaseous or liquid C₃ hydrocarbon stream (e1);
F) optionally feeding said C₃ hydrocarbon stream (e1) into a first distillation zone and distillatively separating said C₃ hydrocarbon stream (e1) into a stream (f1) composed of propane and propene and a stream (f2) comprising ethane and ethene;
G) feeding said stream (e1) or (f1) into a second distillation zone and distillatively separating said stream (e1) or (f1) into a product stream (g1) composed of propene and a stream (g2) composed of propane, wherein said stream (g2) is at least partly recycled into said dehydrogenation zone.

17. The process of claim 16, wherein said dehydrogenation of said propane of said feed gas stream (a) to propene in B) is carried out as an oxidative or nonoxidative dehydrogenation.

18. The process of claim 16, wherein said dehydrogenation of said propane of said feed gas stream (a) to propene in B) is carried out adiabatically or isothermally.

19. The process of claim 16, wherein said dehydrogenation of said propane of said feed gas stream (a) to propene in B) is carried out in a fixed bed reactor, moving bed reactor, or fluidized bed reactor.

20. The process of claim 16, wherein an oxygen-containing gas stream is fed into B).

21. The process of claim 20, wherein said dehydrogenation of said propane of said feed gas stream (a) to propene in B) is carried out as an autothermal dehydrogenation or as an oxydehydrogenation.

22. The process of claim 16, wherein the product gas stream (b) is cooled in C) to a temperature in the range of from 10 to 80° C.

23. The process of claim 16, wherein subsequent to G) said stream (g2) and fresh propane are fed into a third distillation zone and separated distillatively into said feed gas stream (a) and a stream comprising C₄⁺ hydrocarbons.

24. The process of claim 16, wherein said inert absorbent in D) is selected from the group consisting of C₄ to C₁₈ alkanes, naphtha, and the middle oil fraction from paraffin distillation.

25. The process of claim 16, wherein said product gas stream (c) is contacted with said inert absorbent in D) in an absorption column comprising an absorption section and a rectification section, wherein laden inert absorbent is contacted with a stripping gas stream in said rectification section.

26. The process of claim 16, wherein said product gas stream (d) is compressed to a pressure of from 5 to 25 bar in E).

27. The process of claim 16, wherein C₃-hydrocarbon-laden absorption agent is compressed in D) to a pressure of from 5 to 25 bar prior to desorption.

28. The process of claim 16, wherein said product gas stream (d) is cooled to a temperature in the range of from −10 to 60° C. in E).

29. The process of claim 16, wherein an aqueous condensate stream (e2) is additionally obtained in E) and is removed from the liquid C₃ hydrocarbon stream in a phase separation apparatus.

30. The process of claim 16, wherein said oxygenous gas stream in B) is air or oxygen-enriched air having an oxygen content of up to 70% by volume.
